SEQUENCE LISTING (13 sequences)

SEQ ID NO: 1 - DNA, 19 bp, Homo sapiens
cctgctgaaaatgactgaa

SEQ ID NO: 2 - DNA, 15 bp, Homo sapiens
cgatcagacgacgac

SEQ ID NO: 3 - DNA, 19 bp, Homo sapiens
cctgctgaaaatgactgaa

SEQ ID NO: 4 - DNA, 15 bp, Homo sapiens
cgatcagacgacgac

SEQ ID NO: 5 - DNA, 19 bp, Homo sapiens
cctgctgaaaatgactgaa

SEQ ID NO: 6 - DNA, 23 bp, Homo sapiens
gccaccagctccaactaccacaa

SEQ ID NO: 7 - DNA, 24 bp, Homo sapiens
agacctcacagtaaaaataggtga

SEQ ID NO: 8 - DNA, 15 bp, Homo sapiens
cgatcagacgacgac

SEQ ID NO: 9 - DNA, 20 bp, Homo sapiens
catgaagacctcacagtaaa

SEQ ID NO: 10 - DNA, 20 bp, Homo sapiens
gatttcactgtagctagacc

SEQ ID NO: 11 - DNA, 15 bp, Homo sapiens
attattccaagctcc

SEQ ID NO: 12 - DNA, 18 bp, Homo sapiens
agctggtgacgtaataat

SEQ ID NO: 13 - DNA, 19 bp, Homo sapiens
ttctctgtagctagaccaa

(19) United States
(12) Patent Application Publication
(10) Pub. No.: US 2021/0277485 A1
(43) Pub. Date: Sep. 9, 2021

(54) Method for prognosis of colorectal cancer

(71) Applicant: Jakob Stenman, Saltsjö-Boo (SE)

(72) Inventor: Jakob Stenman, Saltsjö-Boo (SE)

(21) Appl. No.: 17/259,943

(22) PCT Filed: Jul. 5, 2019
(86) PCT No.: PCT/FI2019/050534

§ 371 Date: Jan. 13, 2021

(30) Foreign Application Priority Data
Jul. 13, 2018 (FI) 20185643
Sep. 20, 2018 (FI) 20185785

(51) Int. Cl. C12Q 1/6886

(52) U.S. Cl. CPC C12Q 1/6886; C12Q 2600/106; C12Q 2600/156; C12Q 2600/118

(57) ABSTRACT

The invention relates to the prognosis of colorectal cancer, especially left-sided (distal) colorectal cancer. The invention provides an assay method for selecting patients suffering from colorectal cancer for different prognostic groups, the method comprising the steps of detecting mutations in expressed KRAS mRNA and selecting the prognostic group of said patient, wherein the presence or increased level of a mutation in expressed KRAS mRNA indicates that the patient having a left-sided colorectal cancer has a worse prognosis than in the absence of a mutation in expressed KRAS mRNA.

### FIELD OF THE INVENTION

The invention relates to the prognosis of colorectal cancer, especially left-sided (distal) colorectal cancer. It also relates to identifying patients with distal colorectal cancer who are likely to respond to cancer therapy. The present invention is also useful for selecting cancer patients for clinical trials.

### BACKGROUND OF THE INVENTION

Determination of KRAS mutational status has become an integral part of the diagnostic laboratory routines for patients diagnosed with colorectal cancer (CRC). While KRAS mutations are known to confer resistance to treatment with EGFR inhibitors, the prognostic role of KRAS mutations is under debate.

Colorectal cancer (CRC) is the fourth leading cause of cancer-related death worldwide [1], nevertheless, a proportion of CRC patients experience long-term recurrence-free survival. Mutations in the kirsten rat sarcoma viral oncogene homolog (KRAS) are considered to be an early event in the developmental sequence leading to adenoma-carcinoma. KRAS mutations in DNA are, however, detectable approximately in only one-third of CRC tumors, with a large majority located in codons 12 and 13 [2-5]. The KRAS gene encodes for one of the proteins in the epidermal growth factor receptor (EGFR) signaling pathway that is critical in the development and progression of this cancer type. Mutations in KRAS yield a constitutively active protein [6-8], which compromise the hydrolysis of ras-bound GTP to GDP, resulting in constitutive activation of the downstream pathways, such as the MAPK and PI3K/Akt pathways [7-10].

As a standard clinical practice, all CRC patients that are candidates for anti-EGFR therapy are tested for KRAS mutational status, as the association between tumor KRAS mutations and resistance to treatment with anti-EGFR monoclonal antibodies cetuximab and panitumumab has been firmly established [9]. Yet the prognostic relevance of KRAS mutations itself remains controversial [10-14]. Early studies by Michelassi et al., using a monoclonal antibody to detect p21 Ras proteins, reported positive staining to be a prognostic indicator in rectal cancer patients [15], and to correlate with the malignant potential of pre-cancerous lesions and malignant tumors in the colon and rectum [16]. Accordingly, phenotypes of cells or tissues and the prognosis of CRC patients could be associated with the mutation-specific expression of the KRAS gene, which may be differentially expressed in an allele-specific manner [17]. Therefore, we hypothesized that mutation-specific RNA expression of KRAS might potentially have additional diagnostic and prognostic value compared to the sole determination of KRAS mutational status from the DNA. KRAS mutation testing, as it is performed today, might thus overlook highly relevant tumor characteristics that would be conferred by mutation-specific RNA expression of the KRAS gene.

In addition to the KRAS mutations, V-Raf murine sarcoma viral oncogene homolog B1 (BRAF) V600E mutation, which is found in approximately 10% of CRCs, has also been proposed to confer resistance to EGFR-targeted therapies (Dienstmann and Tebernero, Cancer J 2016). The presence of KRAS and BRAF mutations have been considered mutually exclusive (Rajagobalan et al, Nature 2002). However, recent case reports have shown that these mutations co-exist in metastatic CRC (Larki et al, Cell J 2017; Vittal et al, Case Rep Oncol Med 2017; Deshwar et al, Anticancer Res 2018).

WO 2016/189282 discloses a method of treatment and prognosis of colorectal cancer, especially proximal colorectal cancer.

### SUMMARY OF THE INVENTION

The inventors have surprisingly discovered that the specific mRNA expression of KRAS and BRAF mutations in a large cohort of colorectal cancer (CRC) patients show high prognostic value for left-sided CRC. The inventors found that (i) mRNA expression of KRAS and BRAF^{V600E} mutations were observed in 46.6% and 11.4% among CRC patients, respectively, and (ii) mRNA expressions of KRAS mutations strongly correlated with poor prognosis among BRAF wild-type CRC patients; and (iii) the poor prognosis of CRC patients is proportional to the levels of KRAS-mutation containing mRNAs in the sample, and that this correlation is highly significant in left-sided colorectal cancer. Accordingly, an aim of the present invention is to provide an assay method for selecting patients suffering from colorectal cancer for different prognostic groups, the method comprising the steps of:

a) providing information whether the patient's cancer is left-sided or right-sided colorectal cancer;

b) providing a tissue biopsy sample of the cancer or alternatively a liquid biopsy sample, such as blood or plasma sample, from said patient;

c) preparing an RNA sample from said sample of step b);

d) detecting the presence and optionally level of mutated KRAS mRNA in said RNA sample;

e) selecting the prognostic group of said patient based on the results obtained in steps a) and d), wherein the presence of a mutation in expressed KRAS mRNA indicates that the patient having a left-sided colorectal cancer has a worse prognosis than in the absence of a mutation in KRAS mRNA or low level of presence of mutated KRAS mRNA.

Another aim of the present invention is to provide a method for detecting and analyzing whether a patient suffering from a colorectal cancer is responsive or non-responsive to a cancer treatment, the method comprising the steps of:

a) providing the information whether the patient's cancer is left-sided or right-sided colorectal cancer;

b) providing a tissue biopsy sample of the cancer or alternatively a liquid biopsy sample, such as blood or plasma sample, from said patient;

c) preparing an RNA sample from said sample of step b);

d) detecting the presence and optionally level of mutated KRAS mRNA in said RNA sample;

e) selecting said patient for a cancer treatment based on the results obtained in steps a) and d), wherein the presence of a mutation in expressed KRAS mRNA indicates that the patient having a left-sided colorectal cancer is likely responsive or non-responsive to cancer treatments including EGFR, MAPK or pi3k/akt targeted treatments.

The invention also provides a method for detecting the risk of cancer recurrence or cancer death for a patient suffering from a colorectal cancer, the method comprising the steps of:

a) providing the information whether the patient's cancer is left-sided or right-sided colorectal cancer;

b) providing a tissue biopsy sample of the cancer or alternatively a liquid biopsy sample, such as blood or plasma sample, from said patient;

c) preparing an RNA sample from said sample of step b);

d) detecting the presence and optionally level of mutated KRAS mRNA in said RNA sample;

e) determining the risk of cancer recurrence or cancer death for said patient based on the results obtained in steps a) and d), wherein the presence of a mutation in expressed KRAS mRNA indicates that the patient having a left-sided colorectal cancer has higher risk of cancer recurrence or cancer death.

The invention further provides a method for selecting patients for clinical trials testing efficacy of a cancer treatment, the method comprising the steps of:

a) providing the information whether the patient's cancer is left-sided or right-sided colorectal cancer;

b) providing a tissue biopsy sample of the cancer or alternatively a liquid biopsy sample, such as blood or plasma sample, from said patient;

c) preparing an RNA sample from said sample of step b);

d) detecting the presence and optionally level of mutated KRAS mRNA in said RNA sample;

e) selecting the patients for the clinical trial based on the results obtained in steps a) and d), wherein those patients having a left-sided colorectal cancer and having a mutation in expressed KRAS mRNA are selected for clinical trials.

### DESCRIPTION OF THE DRAWINGS

FIG. 1. Side-specific impact of RNA expression of KRAS mutations in tumor tissue on the survival of colorectal cancer patients. Kaplan-Meier survival curves showing disease-specific survival (DSS) for patients with or without RNA expression of KRAS mutations in (A) left-sided and (B) right sided tumors. Tumors expressing BRAF mutations were excluded from the analysis.

FIG. 2. Kaplan-Meier estimate of the disease-specific survival of patients with left-sided colorectal cancer stratified based on the level of mutant KRAS mRNA determined in the tumor tissue sample. Tumors expressing BRAF mutations were excluded from the analysis.

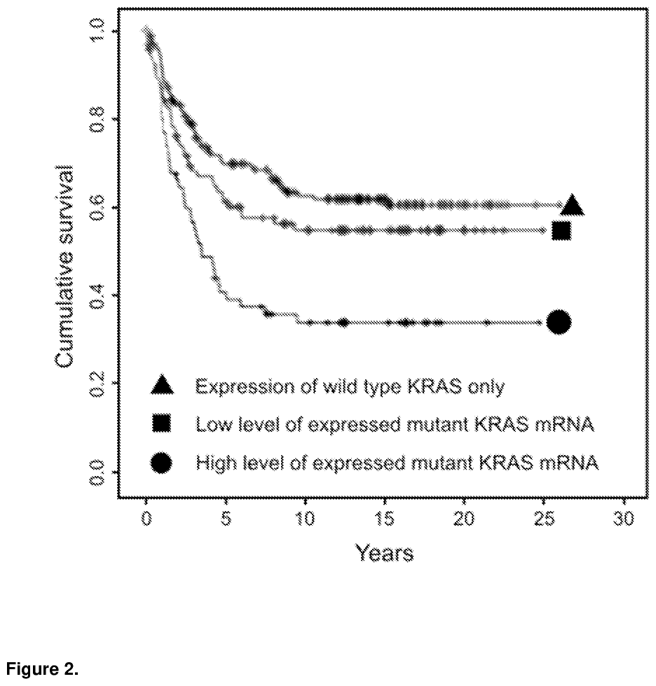

### DETAILED EMBODIMENTS

It is shown in the present invention that RNA expression of KRAS mutations in tumor tissue is a novel powerful predictor of survival in left-sided colorectal cancer. The present invention is thus directed to an assay method for selecting patients suffering from colorectal cancer for different prognostic groups, the method comprising the steps of:

a) providing the information whether the patient's cancer is left-sided or right-sided colorectal cancer;

b) providing a tissue biopsy sample of the cancer or alternatively a liquid biopsy sample, such as blood or plasma sample, from said patient;

c) preparing an RNA sample from said sample of step b);

d) detecting the presence and optionally level of mutated KRAS mRNA in said RNA sample;

e) selecting the prognostic group of said patient based on the results obtained in steps a) and d), wherein the presence of a mutation in expressed KRAS mRNA indicates that the patient having a left-sided colorectal cancer has a worse prognosis than in the absence of a mutation in KRAS mRNA or low level of presence of mutated KRAS mRNA.

In the present description, the phrase “low level of presence of mutated KRAS mRNA” means a measured mutated KRAS mRNA level which does not reach the predictive threshold defined for a patient population. It is well-known in the art how to calculate a predictive threshold for a biomarker (see, e.g., Gosho et al., 2012, Sensors 2012, 12, 8966-8986; doi:10.3390/s120708966).

Preferably, in step e) the prognostic group of said patient is selected based on the results obtained in steps a) and d), wherein an increased level of mutated KRAS mRNA in said sample indicates that a patient having a left-sided colorectal cancer has a worse prognosis than a patient with a low level of, or no mutated KRAS mRNA in said sample.

The colorectal cancer as defined herein preferably is a distal colorectal cancer (or a distal colorectal tumor). The distal colon is the region of the large bowel distal to the splenic flexure, meaning the descending colon, the sigmoid colon and the rectum. Cancers or tumors in this region are also referred to as left-sided cancers or tumors. The invention concerns diagnosis or prognosis of left-sided colorectal cancer or a left-sided colorectal tumor.

The colorectal cancer as defined herein may also be proximal colorectal cancer (or a proximal colorectal tumor). The proximal colon is the region of the large bowel proximal to the splenic flexure, meaning the caecum, the ascending colon and the transverse colon. Cancers or tumors in this region are also referred to as right-sided cancers or tumors. The invention may also concern diagnosis or prognosis of right-sided colorectal cancer or a right-sided colorectal tumor.

The present invention concerns the prognosis of a colorectal cancer or tumor comprising a mutation or mutations in the KRAS and optionally BRAF gene(s). KRAS (Gene ID: 3845; NCBI Reference Sequence: NP_004976.2) is a GTPase which hydrolyses GTP to GDP allowing for activation of a number of downstream signalling pathways including phosphatidyl-inositil and mitogen activated kinase pathways. BRAF (Gene ID: 673; NCBI Reference Sequence: NP_004324.2) is a human gene that encodes a protein called B-Raf also known as serine/threonine-protein kinase B-Raf.

Common mutations in KRAS reduce its intrinsic GTPase function, preventing hydrolysis of GTP to GDP, thus locking KRAS in its active state. This results in constitutive activation of downstream signaling pathways that can drive oncogenesis. In BRAF, mutations relevant to cancer promote the movement of activation loop to the active conformer.

Several KRAS and BRAF mutations are known in the art. A cancer comprises a KRAS or BRAF mutation if one or more of the cells in the cancer comprise(s) a KRAS or BRAF mutation.

Preferably, the KRAS mutations are point mutations at one or more of positions 12, 13, 14, 59, 61, 117, 120, 144, 145 and 146 of the KRAS amino acid sequence. The variant may comprise a point mutation at any number and combination of these positions.

A mutant KRAS gene preferably comprises one or more of the following point mutations G12A, G12C, G12D, G12R, G125, G12V, G13A, G13C, G13D, G13R, G13V, V14I, A59G, Q61H, Q61K, Q61L, Q61R, K117N, L120V, S145T, A146P, A146T, and A146V. The mutant KRAS may comprise any number and combination of the point mutations. The mutant KRAS most preferably comprises one point mutation.

A mutant BRAF gene preferably comprises valine (V) being substituted for by glutamate (E) at codon 600 (referred to as V600E). Other known BRAF mutants are E585K, D593V, F594L, G595R, L596V, T598I, V599D, V599E, V599K, V599R, and V600K. The mutant BRAF may comprise any number and combination of the point mutations. The mutant BRAF most preferably comprises one point mutation.

In the present invention, the presence of one or more KRAS/BRAF point mutations is/are preferably identified using a method as described in WO2013160563 and Ho et al., 2015 [18]. The presence of point mutations is identified using the mRNA encoding the KRAS/BRAF protein in the cancer cells. The method allows the presence or absence of the one or more point mutations to be determined. Preferably, said method comprises i) performing a competitive cDNA synthesis assay comprising a mutation-specific primer specific to a KRAS/BRAF mRNA variant of interest, a second blocking primer specific to KRAS/BRAF wild type sequence, an RNA-dependent DNA polymerase, and RNA from said RNA sample as a template, wherein said mutation-specific primer comprises a sequence specific to said variant of interest and a 5′-tail sequence not complementary to the KRAS/BRAF gene, wherein said 5′-tail generates a priming site for use in the subsequent step;

ii) performing a PCR reaction, preferably a qPCR reaction, comprising a primer specific to said 5′-tail;

iii) detecting the absence or presence and level of amplification reaction products corresponding to said KRAS/BRAF mRNA variant of interest.

In a another embodiment, the method comprises i) performing a competitive cDNA synthesis comprising a first primer specific to a first KRAS mRNA variant, a second primer specific to a second KRAS mRNA variant, an RNA-dependent DNA polymerase, and RNA from said RNA sample as a template, wherein said first primer and said second primer comprise an allele-specific sequence, a target-specific sequence and tag units with a common sequence and/or a discriminating sequence so that the sequence of said tag units is not complementary to said first or second KRAS mRNA variant, wherein each of the cDNA products obtained from said competitive cDNA synthesis consists of the sequence of only one primer extended by the sequence complementary to one of the target KRAS mRNA variants, wherein said first KRAS mRNA variant and said second KRAS mRNA variant are alternative RNA sequences at the same physical locus on a RNA segment;

ii) performing an amplification reaction so that at least part of the cDNA synthesis products obtained from step i) are amplified;

iii) detecting the presence and level of amplification reaction products corresponding to the first and/or second KRAS mRNA variant obtained from step ii) utilizing the presence of the allele-specific sequence or the discriminating sequence of said tag units in said amplification reaction products.

An “allele-specific primer” or “primer specific to an RNA variant” as used herein refers to an oligonucleotide that hybridizes to sequence comprising respective KRAS/BRAF RNA variant of target sequence and initiate cDNA synthesis when placed under conditions, which induce synthesis. Allele-specific primers are specific for a particular KRAS/BRAF RNA variant of a given target sequence and can be designed to detect a difference of as little as one nucleotide in the target sequence. Allele-specific primer comprises a target-specific portion, an allele-specific portion and optionally a tag unit or tag units.

As used herein, the term “target-specific portion” or “target-specific sequence” refers to the region of an allele-specific primer that hybridizes to target sequence. In some embodiments, the target-specific portion of the allele-specific primer may comprise an allele-specific nucleotide portion. In other embodiment, the target-specific portion of the allele-specific primer is adjacent to the 3′ allele-specific nucleotide portion.

As used herein, the term “tag units” refers to one or more nucleotides or non-nucleotide units that are different from a nucleotide fully complementary with corresponding nucleotides on the target sequence when the allele-specific primer stably hybridizes to the sequence comprising the respective KRAS/BRAF RNA variant of target sequence. In some embodiments, the tag units may be located 5′ to the target-specific portion, i.e. the primer comprises a 5′ nucleotide tail. In some other embodiments, the tag units may be located inside the target-specific portion. In some further embodiments, the tag units may be located at both said regions. The tag units may comprise “a common sequence” and “a discriminating sequence”. The common sequence is a portion of identical nucleotide sequence in competitive allele-specific primers used in the same cDNA synthesis. The term “discriminating sequence” refers to the differential portions of nucleotide sequence in the competitive allele-specific primers having essentially different nucleotide sequences in order to facilitate the detection of cDNA products derived from each of the competitive allele-specific primers and enable amplification of said cDNA products using for example separate downstream PCR primers specific to said discriminating sequence in each primer.

As used herein, the term “KRAS/BRAF RNA variant” or “KRAS/BRAF RNA allele” refers generally to alternative RNA sequences at the same physical locus on a KRAS/BRAF RNA segment. A RNA variant can refer to RNA sequences which differ between the same physical locus within a single cell or organism or which differ at the same physical locus in multiple cells or organisms. In some instances, an RNA variant can correspond to a single nucleotide difference at a particular physical locus.

In other embodiments an RNA variant can correspond to more significant difference, such as nucleotide (single or multiple) insertions or deletions.

In another embodiment of the invention, in step d) of the present methods the presence of one or more point mutations may be detected using any of the multiple methods used for detection of point mutations at a sufficient level of sensitivity. The presence of point mutations in the KRAS gene are typically detected in polynucleotides, such as DNA or mRNA, encoding the KRAS protein. Mutations in RNA can be detected and measured using common RNA-sequencing techniques. Mutations in a cDNA template can be detected and measured by allele-specific amplification (ASA) such as allele-specific PCR (ASPCR) or digital droplet PCR (ddPCR). Another commonly used strategy is to amplify a region of interest by PCR amplification from complementary DNA (cDNA) transcribed and isolated from cancer tissue or cells. KRAS/BRAF mutations can then be detected and measured using common techniques for post-amplification detection of point-mutations including sequencing.

The KRAS/BRAF mutation is typically measured in a cancer tissue biopsy obtained from the patient. The tissue biopsy may be formalin fixed paraffin embedded (FFPE) tissue, fresh or frozen tissue. The KRAS/BRAF mutation can alternatively be measured in a sample of body fluid. Detection of the mutational status of a target gene in nucleic acids isolated from a sample of body fluid, such as blood, plasma or urine, is becoming a routine technique in cancer diagnostics. This is called liquid biopsy and it is one of the fastest growing technologies for cancer diagnostics, prognostics, as well as for monitoring response to treatment. The detection method may also be carried out on cancer cells circulating in the blood of the patient or on urinary or blood exosomes. Another suitable sample is isolated blood platelets as they are shown to contain tumor derived RNA biomarkers, see Nilsson et al., 2011 [20] and Best et al., 2015 [19]. The method may also be carried out on a stool sample. The methods of the present invention are thus typically carried out in vitro.

As used herein, the term “increased level of mutated KRAS mRNA” in a sample, refers to the amount of mutated KRAS amplicons that are detected during or after PCR amplification. The level of mutated KRAS mRNA can be determined based on the absolute amount of mutated KRAS PCR amplicons, or in relation to the level of wild type KRAS amplicons derived from the same sample. An increased level of mutated KRAS mRNA can arise either from a high expression level of the mutant allele in the cells harboring the KRAS mutation, or from a high proportion of cells expressing the mutant allele in the sample. The level of mutated KRAS mRNA determined in patient samples can be used to rank patients within a group for risk of recurrence or death, and for likelyhood of response to treatment. The level of mutated KRAS mRNA determined in patient samples can also be used for dividing patients into groups with different prognostic or risk profiles and for selecting patient for clinical trials.

The present method is also directed to a method for detecting and analyzing whether a patient suffering from a colorectal cancer is responsive or non-responsive to a cancer treatment, the method comprising the steps of:

a) providing the information whether the patient's cancer is left-sided or right-sided colorectal cancer;

b) providing a tissue biopsy sample of the cancer or alternatively liquid biopsy sample, such as blood or plasma sample, from said patient;

c) preparing an RNA sample from said sample of step b);

d) detecting the presence and optionally level of mutated KRAS mRNA in said RNA sample;

e) selecting said patient for a cancer treatment based on the results obtained in steps a) and d), wherein the presence of a mutation in expressed KRAS mRNA indicates that the patient having a left-sided colorectal cancer is likely responsive or non-responsive to cancer treatments including EGFR, MAPK or pi3k/akt targeting treatments.

Preferably, in step e) said patient for a cancer treatment is selected based on the results obtained in steps a) and d), wherein an increased level of mutated KRAS mRNA in said sample indicates that the patient having a left-sided colorectal cancer is likely responsive or non-responsive to cancer treatments including EGFR, MAPK or pi3k/akt targeting treatments.

The invention is also directed to a method for detecting the risk of cancer recurrence or cancer death for a patient suffering from a colorectal cancer, the method comprising the steps of:

a) providing the information whether the patient's cancer is left-sided or right-sided colorectal cancer;

b) providing a tissue biopsy sample of the cancer or alternatively a liquid biopsy sample, such as blood or plasma sample, from said patient;

c) preparing a RNA sample from said sample of step b);

d) detecting the presence and optionally level of mutated KRAS mRNA in said RNA sample;

e) determining the risk of cancer recurrence or cancer death for said patient based on the results obtained in steps a) and d), wherein the presence of a mutation in expressed KRAS mRNA indicates that the patient having a left-sided colorectal cancer has higher risk of cancer recurrence or cancer death.

Preferably, in step e) the risk of cancer recurrence or cancer death for said patient is determined based on the results obtained in steps a) and d), wherein an increased level of mutated KRAS mRNA in said sample indicates that the patient having a left-sided colorectal cancer has a higher risk of cancer recurrence or cancer death than a patient with a low level of, or no mutated KRAS mRNA in said sample.

The invention is further directed to a method for selecting patients for clinical trials testing efficacy of a cancer treatment, the method comprising the steps of:

a) providing the information whether the patient's cancer is left-sided or right-sided colorectal cancer;

b) providing a tissue biopsy sample of the cancer or alternatively a liquid biopsy sample, such as blood or plasma sample, from said patient;

c) preparing a RNA sample from said sample of step b);

d) detecting the presence and optionally level of mutated KRAS mRNA in said RNA sample;

e) selecting the patients for the clinical trial based on the results obtained in steps a) and d), wherein those patients having a left-sided colorectal cancer and having a mutation in expressed KRAS mRNA are selected for clinical trials.

Preferably, in step e) the patients for the clinical trial are selected based on the results obtained in steps a) and d), wherein those patients having a left-sided colorectal cancer and having an increased level of mutated KRAS mRNA in said sample are selected for clinical trials.

In the step e) of the all above methods, a person skilled in the art is capable to use a computer-based system for handling the results obtained in the previous steps of the methods. The system may comprise (a) a storage module configured to store control data preferably obtained from prior art and output data from said previous steps, (b) a computation module configured to provide a comparison between the value of the output data obtained and the control data; and (c) an output module configured to display the results in view of the control data.

The publications and other materials used herein to illuminate the background of the invention, and in particular, to provide additional details with respect to its practice, are incorporated herein by reference. The present invention is further described in the following experimental section, which are not intended to limit the scope of the invention.

### ERIMENTAL SECTION

Materials and Methods

RNA Samples

RNA was extracted from FFPE samples from patients who were operated for histologically confirmed CRC at the Department of Surgery, Meilahti Hospital, Helsinki University Hospital between 1987 and 2003. In total, 775 patient's samples were available for this study. The use of clinical samples for this purpose was approved by the Surgical Ethics Committee of Helsinki University Hospital and the National Supervisory Authority of Welfare and Health and collected from the archives of the Department of Pathology, Helsinki University Hospital. All RNA samples were quantified with a NanoVue spectrophotometer (GE Healthcare, Waukesha, Wis., USA). RNA was extracted using phenol-chloroform extraction and stored at −80° C. Before the ExBP-RT reaction, all RNA samples were quantified with a NanoVue spectrophotometer (GE Healthcare, Waukesha, Wis., United States) then diluted to 100 ng/μL in diethyl pyrocarbonate (DEPC) H2O.

RNA extracted from A549, Lovo, and Colo205 cell lines was used as positive and negative controls, respectively (Table 1). All control RNAs were extracted from cell cultures using RNA/DNA purification kit (Norgen Biotek) then quantified with a NanoVue spectrophotometer and diluted to 100 ng/μL in DEPC H2O before using.

TABLE 1
Cell line RNAs were used as positive controls

| | | KRAS Codon | KRAS Codon | BRAF | |
|---|---|---|---|---|---|
| Cell | | 12 mutation | 13 mutation | mutation | |
| line | Mutation | analysis | analysis | analysis | Concentration |
| A549 | KRAS - G12R | Mutant | — | Wild-type | 100 ng/μl |
| | | control | | control | |
| Lovo | KRAS - G13D | — | Mutant | — | 100 ng/μl |
| | | | control | | |
| Colo205 | BRAF - V600E | Wild-type | Wild-type | Mutant | 100 ng/μl |
| | | control | control | control | |

ExBP-RT—Based Mutation Detection Assays

ExBP-RT was employed for ultrasensitive detection of most common mutations of the KRAS and BRAF genes as described previously [18]. Mutations covered in this study include six different common KRAS mutations at codon 12 (G12D, G12A, G12V, G125, G12A, and G12C), one KRAS mutation at codon 13 (G13D) and the BRAF V600E mutation. The principles of ExBP-RT assays and reaction setup procedures for multiplex detection of six KRAS mutations at codon 12 were as described in the original article, as well as the BRAF V600E mutation analyses [18]. ExBP-RT primers and probes are listed in Table 2.

Detection and Quantification of Expressed Mutations

Using products of the ExBP-RT assays as template, real-time PCR amplification was performed to detect/quantify expressed mutations of KRAS. QuantiTect Probe PCR Kits (QIAGEN) were used for these probe-based real-time PCR assays according to the manufacturer's instructions in a 10-μL reaction volume. The Taqman probes for simultaneous detection of six different KRAS mutations at codon 12 were designed to contain two universally binding inosine nucleotides that allows for targeting the variant nucleotides of different types of mutation. A common reverse primer was designed to target the 5′-prime tail of all mutation-specific ExBP-RT products. The expression levels of total KRAS (including mutants and wild-type) were also determined in each sample for normalization using QuantiTect SYBR Green PCR Kits (QIAGEN) according to the manufacturer's instructions in a 10-μL volume. The sequences and concentration of quantitative PCR (qPCR) primers and probes are provided in Table 2. The same thermocycling conditions were used for both probe-based and SYBR-based qPCRs: 95° C. for 15 minutes, 45 cycles at 94° C. for 10 seconds, and at 60° C. for 45 seconds. Following SYBR-based qPCR, the specificity of the amplification products was always verified by melting curve analysis. All qPCR assays were run on a LightCycler 480 II Instrument real-time PCR (Roche Diagnostics Oy, Finland) with 384-well white plate. All mutation controls, wild-type controls, and H2O controls of each experiment were checked to verify the correction of results in both ExBP-RT and qPCR assays and avoid the problem of contamination.

TABLE 2
Primers and probe sequences for qPCR of different ExBP-RT assays
(Locked Nucleic Acid (LNA) = [+A],[+G], [+C], [+T]; Inosine = i; 6-carboxyfluorescein:
FAM; Black Hole Quenchers: BHQ).

| | | |
|---|---|---|
| Primers and probes | Sequences (5′-3′) | Concentrations |

Mutant KRAS codon 12 assays

| | | |
|---|---|---|
| KRAS Forward primer | 5′-CCTGCTGAAAATGACTGAA-3′ (SEQ | 0.5 μM |
| | ID NO: 1) | |
| Common Reverse | 5′-CGATCAGACGACGAC-3′ (SEQ ID | 0.5 μM |
| primer | NO: 2) | |
| KRAS12-Probe | FAM- | 0.1 μM |
| | AT[+T]A[+T]T[+C]C[+A]ii[+A]G[+C]TCC- | |
| | BHQ1 (SEQ ID NO: 11) | |

Mutant KRAS codon 13 assays

| | | |
|---|---|---|
| KRAS Forward primer | 5′-CCTGCTGAAAATGACTGAA-3′ (SEQ | 0.5 μM |
| | ID NO: 3) | |
| Common Reverse | 5′-CGATCAGACGACGAC-3′ (SEQ ID | 0.5 μM |
| primer | NO: 4) | |
| KRAS13-Probe | FAM- | 0.1 μM |
| | AGC[+T]GG[+T]GA[+C]G[+T]AA[+T]AAT- | |
| | BHQ1 (SEQ ID NO: 12) | |

Total-KRAS assays

| | | |
|---|---|---|
| KRAS Forward primer | 5′-CCTGCTGAAAATGACTGAA-3′ (SEQ | 1.5 μM |
| | ID NO: 5) | |
| Total KRAS Reverse | 5′-GCCACCAGCTCCAACTACCACAA-3′ | 1.5 μM |
| primer | (SEQ ID NO: 6) | |

Mutant BRAF assays

| | | |
|---|---|---|
| BRAF Forward primer | 5′-AGACCTCACAGTAAAAATAGGTGA- | 0.5 μM |
| | 3′ (SEQ ID NO: 7) | |
| Common Reverse | 5′-CGATCAGACGACGAC-3′ (SEQ ID | 0.5 μM |
| primer | NO: 8) | |
| BRAF-Probe | FAM- | 0.1 μM |
| | TTC[+T]CT[+G]TA[+G]CT[+A]GACCAA- | |
| | BHQ1 (SEQ ID NO: 13) | |

Total-BRAF assays

| | | |
|---|---|---|
| Total BRAF Forward | 5′-CATGAAGACCTCACAGTAAA-3′ | 1.5 μM |
| primer | (SEQ ID NO: 9) | |
| Total BRAF Reverse | 5′-GATTTCACTGTAGCTAGACC-3′ | 1.5 μM |
| primer | (SEQ ID NO: 10) | |

We calculated threshold cycle (Ct) values of qPCR automatically using the absolute quantification analysis with the fit points method, which is built in the LightCycler 480 II system (Roche Diagnostics Oy). That allows to set the noise band and the threshold line to discard uninformative background noise.

Statistical Methods

Results are given as mean and standard deviation (SD) or median and interquartile range (IQR) or number of patients and percentage. Differences in continuous variables between the groups are tested with the non-parametric Mann-Whitney test and in dichotomous or ordinal variables with the Fisher's exact test or the Linear by linear association test. The Kaplan-Meier method and the Cox proportional hazard model were used to analyze survival data. Multivariate model was adjusted for age, gender and tumor location (rectum/colon). Interactions were considered with the Bonferroni correction for multiple testing. The Cox model assumption of constant hazard ratio over time was tested by plotting the scaled Schoenfeld residuals with time and testing relationship between residuals and time. No significant deviation from the assumption was detected. Statistical analyses were performed with R (v.3.4.3, Foundation for Statistical Computing, Vienna, Austria, survival package), and SPSS (v. 24;IBM, New York, USA).

Results

1. RNA Expression of KRAS and BRAF Mutations in Colorectal Cancer

Among the study cohort of 571 colorectal cancer patients, our ExBP-RT assay detected RNA expression of KRAS mutations in 259 patients (45.4%) and expression of the BRAF^{V600E} mutation in 64 patients (11.2%). Co-expression of KRAS and BRAF^{V600E} mutations in RNA was observed in 12 cases (2.1%), with one patient having high level of the KRAS mutation specific RNA expression (0.18%), and whom died only 3.8 months after diagnosis.

There was no significant difference regarding frequencies of KRAS mutations between tumors in left-sided colon compared with right-sided colon (47.2% vs 41.8%; p=0.25). In contrast, RNA expression of the BRAF^{V600E} mutation was much more frequent in tumors located in right-sided CRC as compared with left-sided disease (20.4% vs 6.3%; p <0.001).

RNA expression of KRAS mutations at codon 12 accounted for 36.1% (206/571) and that of KRAS mutation at codon 13 (G13D) accounted for 10.7% (61/571). A high level of the KRAS mutation-specific RNA expression, which was defined as a ΔCt_{mt-wt} value of less than 10 compared with the wild type KRAS RNA expression, was observed in 45.6% of patients with KRAS mutations at codon 12 (94/206) and 34.4% of patients with KRAS G13D mutation (21/61).

2. RNA Expressions of BRAF and KRAS Mutations and Patient Survival

After a median follow-up of 5.9 years, the 5-year disease-specific survival rate (DSS) of the study population was 62.2%, 95% Cl 58.0-66.3%. DSS was significantly worse in patients with RNA expression of KRAS mutations than in patients expressing wild type KRAS RNA only (5 year DSS rate: 50.0% [95% Cl 40.4-59.5%] vs 65.3 [95% Cl 60.7-69.8%]; log rank p=0.06; HR 1.30 [95% Cl 1.01-1.67], p=0.044). There was no significant difference in DSS between patients with RNA expression of KRAS mutations at codon 12 in comparison to codon 13 (5 year DSS rate: 57.8% [95% Cl 50.6-65.0%] vs 55.0% [95% Cl 41.3-68.7%]; log rank p=0.978; HR 0.98, [95% Cl 0.63-1-56], p=0.978).

As expected, DSS was significantly worse in patients with RNA expression of the BRAF^{V600E} mutation than in those expressing wildtype BRAF only (5 year DSS rate: 42.3% [95% Cl 29.7-54.9%] vs 64.7% [95% Cl 60.4-69.1%]; log rank p <0.001; HR 2.10 [95% Cl 1.47-3.00], p <0.001). Considering RNA expression of BRAF^{V600E} and KRAS mutations together, DSS were significantly more favorable in patients with RNA expression of wild-type KRAS and BRAF only, compared to DSS of patients with RNA expression of either KRAS or BRAF^{V600E} mutations (5 year DSS rate: 71.1% [95% Cl 65.3-76.8%] vs 54.8% [95% Cl 49.1-60.6%]; log rank p <0.001). (HR 1.65 [95% Cl 1.26-2.16], p <0.001).

Since BRAF^{V600E} mutations have been well-established as a negative prognostic biomarker[10] and KRAS and BRAF mutations have been considered mutually exclusive, mutations of BRAF^{V600E} represents a significant molecular confounder in survival analysis of patients with wild-type KRAS[11]. In order to study the prognostic value of KRAS mutation-specific RNA expression, independent of BRAF^{V600E} RNA expression, we excluded those patients expressing the BRAF_{V600E} mutation in further analyses. Among patients expressing wild-type BRAF only, the DSS of patients with RNA expression of KRAS mutations was significantly worse than that of patients expressing wild-type KRAS only (5 year DSS rate: 58.1% [95% Cl 51.7-64.6%] vs 71.1% [95% Cl 65.3-76.8%]; log rank p=0.005) (HR 1.49 [95% Cl 1.12-1.98], p=0.006).

3. The Impact of KRAS-Mutated RNA Expression on Survival is Side-Specific and Dose-Dependent

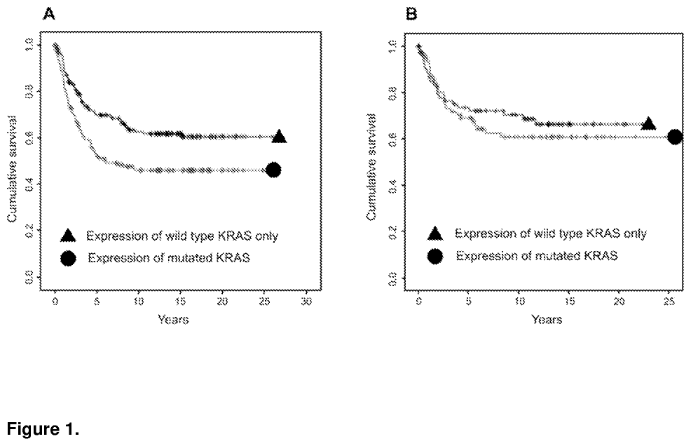

RNA expression of the KRAS mutation had a profound impact on the prognosis of BRAF wild-type patients with tumors on left-sided colon, with significantly decreased DSS (5 year DSS rate: 52.7% [95% Cl 44.8-60.6%] vs 69.9% [95% Cl 62.9-76.9%]; log rank p=0.002; HR 1.66 [95% Cl 1.19-2.31], p=0.003), FIG. 1A. Similar effect was not detected in patients with tumors on right-sided colon (5 year DSS rate: 67.3% [95% Cl 56.9-77.7%] vs 66.7% [95% Cl 57.7-75.7%]; log rank p=0.972; HR 0.99 [95% Cl 0.62-1.59], p=0.972), FIG. 1B.

In order to explore the association between survival and the RNA expression level of KRAS-mutations, we divided study subjects in whom RNA expression of KRAS mutations was detected into two groups, based on the relative expression level of mutant and wild-type KRAS RNA. High expression of KRAS mutations corresponding to a delta Ct_{mt-wt}<10 and low expression corresponding to delta Ct_{mt-wt}>=10, wherein mt is mutant type KRAS/BRAF and wt is wild type KRAS/BRAF. We compared the survival of patients with high, low or no RNA expression of KRAS mutations for each side separately. Among patients with right-sided colon tumors, the prognosis were not statistically different between high-, low- and no-expression of KRAS-mutation RNA (5 year DSS rate: 65.5% [95% Cl 50.8-80.2%], 73.0% [95% Cl 57.9-88.1%] and 73.6% [95% Cl 63.4-83.8%]; log rank p=0.726; HR for high expression 1.29 [95% Cl 0.68-2.41], p=0.432 and HR for low expression 1.06 [95% Cl 0.52-2.18], p=0.868 vs. no expression). In contrast, the difference in DSS between patients with high-, low- and no-expression of KRAS-mutation RNA was highly significant for those with left-sided tumors (5 year DSS rate: 40.8% [95% Cl 28.7-52.9%], 61.3% [95% Cl 51.2-71.4%] and 69.9% [95% Cl 62.9-76.9%]; p<0.001; HR for high expression 2.27 [95% Cl 1.53-3.36], p<0.001 and HR for low expression 1.30 [95% Cl 0.87-1.93], p=0.200 vs. no expression). Among patients with high expression levels of KRAS-mutated RNA, the prognosis of patients with KRAS mutation at codon 13 apparently trended toward being worse than that of patients with KRAS mutation at codon 12 (33.3% vs 51.6%); however, the difference was not statistically significant (p=0.18).

4. RNA Expression of KRAS Mutations and Survival of Left-Sided CRC Patients in Strata of Other Variables

Among CRC patients with left-sided tumors, high-level RNA expression of KRAS mutations is more frequently detected in CRC patients with Duke D stage in comparison to other non-metastasis stages of Duke A, B and C (31,6% vs 11.8%, 19.4% and 16.7%). Similarly, Duke D stage is significantly enriched in the patients with high-level RNA expression of KRAS mutations than in patients with low or no expression of KRAS-mutation RNAs (34.3% vs 18.8%, p=0.0091), which partly explains the negative impact of KRAS-mutation RNA expression on survival. Stratification according to Duke stages revealed negative prognostic impact of KRAS-mutation RNA expression to be clearly distinct for Duke C stage, with the 5 year DSS rates for high-, low- and no-expression of KRAS-mutation RNA expression are 13.3%, 48.1% and 64.6%; HR for high expression 3.89 [95% Cl 1.87-8.08], p =0.0003 and HR for low expression 1.83 [95% Cl 0.9-3.71], p=0.096 vs. no expression. The negative prognostic value of the KRAS-mutation RNA expression was confirmed in subsequent multivariable analysis limited to left-sided CRC patients with Duke C stage (HR for high expression 3.18 [95% Cl 1.48-6.87], p=0.0032 and HR for low expression 1.91 [95% Cl 0.93-3.94], p=0.0788 vs. no expression).

Discussion

Armed with recently published ExBP-RT assay[18] for gene mutation analysis at RNA level, the inventors were capable of measuring the specific RNA expression of KRAS and BRAF mutations in a large cohort of CRC patients and showing here the prognostic value of this novel class of biomarkers. The inventors found that (i) RNA expression of KRAS and BRAF^{V600E} mutations were observed in 46.6% and 11.4% among CRC patients, respectively and that the expression of these mutations were not totally mutually exclusive, correlating with previously published data; (ii) RNA expression of KRAS and BRAF^{V600E} mutations were both associated with poor survival, and RNA expressions of KRAS mutations strongly correlated with worse prognosis among BRAF wild-type CRC patients; and (iii) the poor prognosis of CRC patients is proportional to the expression levels of KRAS-mutation RNAs (with poor-intermediate—good prognosis corresponding to high level-, low level-, and no RNA expression of KRAS mutations), and that this correlation is highly, albeit only, significant in left-sided colorectal cancer.

### REFERENCES

1. Siegel, R., et al., Cancer statistics, 2011: the impact of eliminating socioeconomic and racial disparities on premature cancer deaths. C A Cancer J Clin, 2011. 61(4): p. 212-36.

2. Andreyev, H. J., et al., Kirsten ras mutations in patients with colorectal cancer: the multicenter “RASCAL” study. J Natl Cancer Inst, 1998. 90(9): p. 675-84.

3. Bos, J. L., ras oncogenes in human cancer: a review. Cancer Res, 1989. 49(17): p. 4682-9.

4. Vogelstein, B., et al., Genetic alterations during colorectal-tumor development. N Engl J Med, 1988. 319(9): p. 525-32.

5. Van Cutsem, E., et al., Cetuximab and chemotherapy as initial treatment for metastatic colorectal cancer. N Engl J Med, 2009. 360(14): p. 1408-17.

6. Brink, M., et al., K-ras oncogene mutations in sporadic colorectal cancer in The Netherlands Cohort Study. Carcinogenesis, 2003. 24(4): p. 703-10.

7. Ogino, S., et al., Molecular correlates with MGMT promoter methylation and silencing support CpG island methylator phenotype-low (ClMP-low) in colorectal cancer. Gut, 2007. 56(11): p. 1564-71.

8. Samowitz, W. S., et al., Relationship of Ki-ras mutations in colon cancers to tumor location, stage, and survival: a population-based study. Cancer Epidemiol Biomarkers Prey, 2000. 9(11): p. 1193-7.

9. Lievre, A. et al. KRAS mutation status is predictive of response to cetuximab therapy in colorectal cancer. Cancer Res 66, 3992-3995, doi:66/8/3992 [pii]1158/0008-5472.CAN-06-0191 (2006).

10. Roth, A. D., et al., Prognostic role of KRAS and BRAF in stage II and III resected colon cancer: results of the translational study on the PETACC-3, EORTC 40993, SAKK 60-00 trial. J Clin Oncol, 2010. 28(3): p. 466-74.

11. Imamura, Y., et al., Specific mutations in KRAS codons 12 and 13, and patient prognosis in 1075 BRAF wild-type colorectal cancers. Clin Cancer Res, 2012. 18(17): p. 4753-63.

12. Blons, H., et al., Prognostic value of KRAS mutations in stage III colon cancer: post hoc analysis of the PETACC8 phase III trial dataset. Ann Oncol, 2014. 25(12): p. 2378-85.

13. Lee, D.W., et al., KRAS mutation is associated with worse prognosis in stage III or high-risk stage II colon cancer patients treated with adjuvant FOLFOX. Ann Surg Oncol, 2015. 22(1): p. 187-94.

14. Ogino, S., et al., KRAS mutation in stage III colon cancer and clinical outcome following intergroup trial CALGB 89803. Clin Cancer Res, 2009. 15(23): p. 7322-9.

15. Michelassi, F., et al., Ras oncogene expression as a prognostic indicator in rectal adenocarcinoma. J Surg Res, 1988. 45(1): p. 15-20.

16. Michelassi, F., et al., Ras oncogene p21 levels parallel malignant potential of different human colonic benign conditions. Arch Surg, 1987. 122(12): p. 1414-6.

17. Yan, H., et al., Allelic variation in human gene expression. Science, 2002. 297(5584): p. 1143-1143.

18. Ho, T.H., et al., Extendable blocking probe in reverse transcription for analysis of RNA variants with superior selectivity. Nucleic Acids Res, 2015. 43(1): p. e4 19. Best et al., RNA-Seq of Tumor-Educated Platelets Enables Blood-Based Pan-Cancer, Multiclass, and Molecular Pathway Cancer Diagnostics, Cancer Cell. 2015 Nov 9; 28(5): 666-676.

20. Nilsson et al., Blood platelets contain tumor-derived RNA biomarkers, Blood, 2011, Sep. 29; 118(13): 3680-3683.

1. An assay method for selecting patients suffering from colorectal cancer for different prognostic groups, the method comprising the steps of:
a) providing the information whether the patient's cancer is left-sided or right-sided colorectal cancer;
b) providing a tissue biopsy sample of the cancer or alternatively a liquid biopsy sample, from said patient;
c) preparing an RNA sample from said sample of step b);
d) detecting the presence and optionally level of mutated KRAS mRNA in said RNA sample; and
e) selecting the prognostic group of said patient based on the results obtained in steps a) and d), wherein the presence of a mutation in expressed KRAS mRNA indicates that the patient having a left-sided colorectal cancer has a worse prognosis than in the absence of a mutation in KRAS mRNA or low level of presence of mutated KRAS mRNA.

2. The method according to claim 1, wherein in step e) the prognostic group of said patient is selected based on the results obtained in steps a) and d), wherein an increased level of mutated KRAS mRNA in said sample indicates that a patient having a left-sided colorectal cancer has a worse prognosis than a patient with a low level of, or no mutated KRAS mRNA in said sample.

3. The method according to claim 1, wherein the KRAS mutations detected in step d) resides at codon 12 or 13.

4. The method according to claim 3, wherein the KRAS mutations detected in step d) corresponds to a mutation in codon 12 selected from the group consisting of: G12D, G12A, G12V, G12S, G12A, and G12C.

5. The method according to claim 3, wherein the KRAS mutations detected in step d) corresponds to the mutation G13D.

6. The method according to claim 1, wherein step d) further comprises detecting mutations in expressed BRAF mRNA in said RNA sample.

7. The method according to claim 6, wherein the BRAF mutation detected corresponds to the mutation V600E.

8. The method according to claim 1, wherein step d) is performed by allele-specific cDNA synthesis of KRAS and optionally BRAF sequences, by allele-specific amplification, by techniques for post-amplification detection of point-mutations or by RNA sequencing.

9. The method according to claim 8, wherein step d) comprises:
i) performing a competitive cDNA synthesis assay comprising a mutation-specific primer specific to a KRAS mRNA variant of interest, a second blocking primer specific to KRAS wild type sequence, an RNA-dependent DNA polymerase, and RNA from said RNA sample as a template, wherein said mutation-specific primer comprises a sequence specific to said variant of interest and a 5′-tail sequence not complementary to the KRAS gene, wherein said 5′-tail generates a priming site for use in the subsequent step;
ii) performing a PCR reaction comprising a primer specific to said 5′-tail; and
iii) detecting the absence or presence and level of amplification reaction products corresponding to said KRAS mRNA variant of interest.

10. The method according to claim 9, wherein step d) comprises:
i) performing a competitive cDNA synthesis comprising a first primer specific to a first KRAS mRNA variant, a second primer specific to a second KRAS mRNA variant, an RNA-dependent DNA polymerase, and RNA from said RNA sample as a template, wherein said first primer and said second primer comprise an allele-specific sequence, a target-specific sequence and tag units with a common sequence and/or a discriminating sequence so that the sequence of said tag units is not complementary to said first or second KRAS mRNA variant, wherein each of the cDNA products obtained from said competitive cDNA synthesis consists of the sequence of only one primer extended by the sequence complementary to one of the target KRAS mRNA variants, wherein said first KRAS mRNA variant and said second KRAS mRNA variant are alternative RNA sequences at the same physical locus on a RNA segment;
ii) performing an amplification reaction so that at least part of the cDNA synthesis products obtained from step i) are amplified; and
iii) detecting the presence and level of amplification reaction products corresponding to the first and/or second KRAS mRNA variant obtained from step ii) utilizing the presence of the allele-specific sequence or the discriminating sequence of said tag units in said amplification reaction products.

11-18. (canceled)

19. A method for detecting the risk of cancer recurrence or cancer death for a patient suffering from a colorectal cancer, the method comprising the steps of:
a) providing the information whether the patient's cancer is left-sided or right-sided colorectal cancer;
b) providing a tissue biopsy sample of the cancer or alternatively a liquid biopsy sample from said patient;
c) preparing an RNA sample from said sample of step b);
d) detecting the presence and optionally level of mutated KRAS mRNA in said RNA sample; and
e) determining the risk of cancer recurrence or cancer death for said patient based on the results obtained in steps a) and d), wherein the presence of a mutation in expressed KRAS mRNA indicates that the patient having a left-sided colorectal cancer has higher risk of cancer recurrence or cancer death.

20. The method according to claim 19, wherein in step e) the risk of cancer recurrence or cancer death for said patient is determined based on the results obtained in steps a) and d), wherein an increased level of mutated KRAS mRNA in said sample indicates that the patient having a left-sided colorectal cancer has a higher risk of cancer recurrence or cancer death than a patient with a low level of, or no mutated KRAS mRNA in said sample.

21. The method according to claim 19, wherein the KRAS mutations detected in step d) resides at codon 12 or 13.

22. The method according to claim 21, wherein the KRAS mutations detected in step d) corresponds to a mutation in codon 12 selected from the group consisting of: G12D, G12A, G12V, G12S, G12A, and G12C.

23. (canceled)

24. (canceled)

25. The method according to claim 19, wherein step d) is performed by allele-specific cDNA synthesis of KRAS and optionally BRAF sequences, by allele-specific amplification, by techniques for post-amplification detection of point-mutations or by RNA sequencing.

26. The method according to claim 25, wherein step d) comprises:
i) performing a competitive cDNA synthesis assay comprising a mutation-specific primer specific to a KRAS mRNA variant of interest, a second blocking primer specific to KRAS wild type sequence, an RNA-dependent DNA polymerase, and RNA from said RNA sample as a template, wherein said mutation-specific primer comprises a sequence specific to said variant of interest and a 5′-tail sequence not complementary to the KRAS gene, wherein said 5′-tail generates a priming site for use in the subsequent step;
ii) performing a PCR reaction comprising a primer specific to said 5′-tail; and
iii) detecting the absence or presence and level of amplification reaction products corresponding to said KRAS mRNA variant of interest.

27. The method according to claim 25, wherein step d) comprises:
i) performing a competitive cDNA synthesis comprising a first primer specific to a first KRAS mRNA variant, a second primer specific to a second KRAS mRNA variant, an RNA-dependent DNA polymerase, and RNA from said RNA sample as a template, wherein said first primer and said second primer comprise an allele-specific sequence, a target-specific sequence and tag units with a common sequence and/or a discriminating sequence so that the sequence of said tag units is not complementary to said first or second KRAS mRNA variant, wherein each of the cDNA products obtained from said competitive cDNA synthesis consists of the sequence of only one primer extended by the sequence complementary to one of the target KRAS mRNA variants, wherein said first KRAS mRNA variant and said second KRAS mRNA variant are alternative RNA sequences at the same physical locus on a RNA segment;
ii) performing an amplification reaction so that at least part of the cDNA synthesis products obtained from step i) are amplified; and
iii) detecting the presence and level of amplification reaction products corresponding to the first and/or second KRAS mRNA variant obtained from step ii) utilizing the presence of the allele-specific sequence or the discriminating sequence of said tag units in said amplification reaction products.

28. A method for selecting patients for clinical trials testing efficacy of a cancer treatment, the method comprising the steps of:
a) providing the information whether the patient's cancer is left-sided or right-sided colorectal cancer;
b) providing a tissue biopsy sample of the cancer or alternatively a liquid biopsy sample from said patient;
c) preparing an RNA sample from said sample of step b);
d) detecting the presence and optionally level of mutated KRAS mRNA in said RNA sample; and
e) selecting the patients for the clinical trial based on the results obtained in steps a) and d), wherein those patients having a left-sided colorectal cancer and having a mutation in expressed KRAS mRNA are selected for clinical trials.

29-37. (canceled)
